(19) United States
(12) Patent Application Publication
(10) Pub. No.: US 2004/0015040 A1
(43) Pub. Date: Jan. 22, 2004

(54) Flexible, torsionable cardiac framework for heart wall actuation of the natural heart

(75) Inventor: David Boyd Melvin, Loveland, OH (US)

Correspondence Address: WOOD, HERRON & EVANS, LLP, 2700 CAREW TOWER, 441 VINE STREET, CINCINNATI, OH, 45202, US

(73) Assignee: The University of Cincinnati, Cincinnati, OH

(21) Appl. No.: 10/197,765

(22) Filed: Jul. 18, 2002

(51) Int. Cl. A61N 1/362

(52) U.S. Cl. 600/16

(57) ABSTRACT

An actuation system for assisting the operation of a natural heart is disclosed. The actuation system includes a framework for interfacing with the natural heart and an actuator mechanism that can be coupled to the framework. The framework includes internal framework and external framework elements. The actuator mechanism is operable for deforming at least one framework element for varying the shape of the heart. The framework includes a set of interconnected, passive, intracardiac and extracardiac elements and their interconnections that deform, bend and/or twist in response to movements induced by the actuation system. Some or all of these elements, and the connections between them, are specifically intended to be flexible, in that they may be bent or twisted by means of motion induced by an associated mechanical actuation system.

### RELATED APPLICATIONS

This application is related to an application entitled “A Protective Sheath Apparatus and Method For Use With A Heart Wall Actuation System For The Natural Heart”, filed on even date herewith, which is incorporated herein by reference in its entirety.

### FIELD OF THE INVENTION

This invention relates generally to assisting the natural heart in operation and, more specifically, to actuating the natural heart utilizing a framework coupled to the heart.

### BACKGROUND OF THE INVENTION

The natural human heart and accompanying circulatory system are critical components of the human body and systematically provide the needed nutrients and oxygen for the body. As such, the proper operation of the circulatory system, and particularly, the proper operation of the heart, are critical in the overall health and well-being of a person. A physical ailment or condition which compromises the normal and healthy operation of the heart can therefore be particularly critical and may result in a condition which must be medically remedied.

Specifically, the natural heart, or rather the cardiac tissue of the heart, can fail for various reasons to a point where the heart can no longer provide sufficient circulation of blood for the body so that life can be maintained. To address the problem of a failing natural heart, solutions are offered to provide ways in which circulation of blood might be maintained.

Some solutions involve replacing the heart. Other solutions are directed to maintaining operation of the existing heart. One such solution has been to replace the existing natural heart in a patient with an artificial heart or a ventricular assist device. In using artificial hearts and/or assist devices, a particular problem stems from the fact that the materials used for the interior lining of the chambers of an artificial heart are in direct contact with the circulating blood. Such contact may enhance undesirable clotting of the blood, may cause a build-up of calcium, or may otherwise inhibit the blood's normal function. As a result, thromboembolism and hemolysis may occur. Additionally, the lining of an artificial heart or a ventricular assist device can crack, which inhibits performance, even when the crack is at a microscopic level. Such drawbacks have limited use of artificial heart devices to applications having too brief of a time period to provide a real lasting benefit to the patient.

An alternative procedure also involves replacement of the heart and includes a transplant of a heart from another human or animal into the patient. The transplant procedure requires removing an existing organ (i.e. the natural heart) from the patient for substitution with another organ (i.e. another natural heart) from another human, or potentially, from an animal. Before replacing an existing organ with another, the substitute organ must be “matched” to the recipient, which, at best, can be difficult, time consuming, and expensive to accomplish. Furthermore, even if the transplanted organ matches the recipient, a risk exists that the recipient's body will still reject the transplanted organ and attack it as a foreign object. Moreover, the number of potential donor hearts is far less than the number of patients in need of a natural heart transplant. Although use of animal hearts would lessen the problem of having fewer donors than recipients, there is an enhanced concern with respect to the rejection of the animal heart.

In an effort to continue use of the existing natural heart of a patient, attempts have been made to wrap skeletal muscle tissue around the natural heart to use as an auxiliary contraction mechanism so that the heart may pump. As currently used, skeletal muscle cannot alone typically provide sufficient and sustained pumping power for maintaining circulation of blood through the circulatory system of the body. This is especially true for those patients with severe heart failure.

Another system developed for use with an existing heart for sustaining the circulatory function and pumping action of the heart is an external bypass system, such as a cardiopulmonary (heart-lung) machine. Typically, bypass systems of this type are complex and large, and, as such, are limited to short term use, such as in an operating room during surgery, or when maintaining the circulation of a patient while awaiting receipt of a transplant heart. The size and complexity of bypass systems effectively prohibit their use as a long term solution, as they are rarely portable devices. Furthermore, long term use of a heart-lung machine can damage the blood cells and blood borne products, resulting in post surgical complications such as bleeding, thromboembolism function, and increased risk of infection.

Still another solution for maintaining the existing natural heart as the pumping device involves enveloping a substantial portion of the natural heart, such as the entire left and right ventricles, with a pumping device for rhythmic compression. That is, the exterior wall surfaces of the heart are contacted and the heart walls are compressed to change the volume of the heart and thereby pump blood out of the chambers. Although somewhat effective as a short term treatment, the pumping device has not been suitable for long term use.

Typically, with such compression devices, heart walls are concentrically compressed. A vacuum pressure is then needed to overcome cardiac tissue/wall stiffness, so that the heart chambers can return to their original volume and refill with blood. This “active filling” of the chambers with blood limits the ability of the pumping device to respond to the need for adjustments in the blood volume pumped through the natural heart, and can adversely affect the circulation of blood to the coronary arteries. Furthermore, natural heart valves between the chambers of the heart and leaching into and out of the heart are quite sensitive to cardiac wall and annular (valve ring) distortion. The compressive movement patterns that reduce a chamber's volume and distort the heart walls may not necessarily facilitate valve closure (which can lead to valve leakage).

Therefore, mechanical pumping of the heart, such as through mechanical compression of the ventricles, must address these issues and concerns in order to establish the efficacy of long term mechanical or mechanically assisted pumping. Specifically, the ventricles must rapidly and passively refill at low physiologic pressures, and the valve functions must be physiologically adequate. Also, the myocardial blood flow of the heart must not be impaired by the mechanical device. Still further, pressure independence between the left and right ventricles must be maintained within the heart.

The present invention addresses the issues of heart wall stiffness and the need for active refilling by assisting in the bending (i.e., indenting, flattening, twisting, etc.) of the heart walls, rather than concentrically compressing the heart walls. Because of the mechanics of deformation in hearts having proportions typical in heart failure (specifically, wall thickness/chamber radius ratios), the deformation from bending and the subsequent refilling of the heart requires significantly less energy than would the re-stretching of a wall that has been shortened to change the chamber volume a similar amount. The present invention facilitates such desirable heart wall bending and specifically protects the heart wall during such bending.

Another major obstacle with long term use of such pumping devices is the deleterious effect of forceful contact of different parts of the living internal heart surface (endocardium), one against another, due to lack of precise control of wall actuation. In certain cases, this coaptation of endocardium tissue is probably necessary for a device that encompasses both ventricles to produce independent output pressures from the left and right ventricles. However, it can compromise the integrity of the living endothelium.

Mechanical ventricular wall actuation has shown promise, despite the issues noted above. As such, devices have been invented for mechanically assisting the pumping function of the heart, and specifically for externally actuating a heart wall, such as a ventricular wall, to assist in such pumping functions.

Specifically, U.S. Pat. No. 5,957,977, which is incorporated herein by reference in its entirety, discloses an actuation device for the natural heart utilizing internal and external support structures. That patent discloses an internal and external framework mounted internally and externally with respect to the natural heart, and an actuator element or activator mounted to the framework for providing cyclical forces to deform one or more walls of the heart, such as the left ventricular wall. The invention of U.S. patent application Ser. No. 09/850,554 which is incorporated herein by reference in its entirety further adds to the art of U.S. Pat. No. 5,957,977 and specifically sets forth various embodiments of activators or actuator devices which are suitable for deforming the heart walls and supplementing and/or providing the pumping function for the natural heart.

It is desirable to further improve upon and add to the art and to utilize framework components operably coupled to the heart for providing actuation of the heart to assist its operation.

Accordingly, it is an objective of the present invention to provide a device and method for actively assisting the natural human heart in its operation.

It is still another objective of the present invention to actuate and assist the heart at a proper natural rate in a way suitable for long term usage.

It is another objective of the present invention to assist the heart while allowing one or more of the heart chambers to rapidly and passively refill at low pressure after the actuating device has completed an actuation stroke.

It is a further objective of the present invention to do so while providing different independent pressures on the left and right side of the natural heart.

It is a still further objective of the invention to assist the heart in a way which minimizes damage to the coronary circulation and the lining tissue or endocardium of the heart.

It is another objective of the present invention to assist the heart while maintaining the competence of the heart valves in their natural function.

These objectives and other objectives and advantages of the present invention will be set forth and will become more apparent in the description of the invention below.

### SUMMARY OF THE INVENTION

The present invention addresses the above objectives and other objectives and provides an actuation system for assisting the operation of a natural heart. The actuation system includes a framework for interfacing with the natural heart. The framework includes framework elements such as an internal framework element and an external framework element or multiple such internal and external elements. The actuation system also includes a actuator mechanism coupled to the framework and operable for deforming at least one framework element for varying the shape of the heart. The actuator mechanism is selectively movable between an actuated state and a relaxed state and operable, when in the actuated state, to assume a predetermined shape and thereby indent a portion of the heart wall to effect a reduction in the volume of the heart.

In one preferred embodiment, at least one of the individual components of the framework is flexible for being deformed to induce deformation in at least one of the ventricles of the heart. The entire framework may optionally be made of materials specifically intended to be flexible.

In one embodiment, the internal framework element of the framework includes a first ring configured for placement adjacent one of the valve annuli of the heart, a second ring configured for placement adjacent another of the valve annuli of the heart, and a septal splint configured for coupling to a portion of a septal wall between chambers of the heart.

In another embodiment, the external framework element acts as a yoke for placement around a portion of the exterior surface of the heart and includes a basal arc and a ventricular arc. The basal arc and the ventricular arc can be manufactured as a single, unitary, contiguous band, or as two separate, connected elements.

In another embodiment, the external framework element includes a flexible, non-expandable sheath configured for placement adjacent a low-pressure ventricle of the heart. The margins of the sheath are fixed to the ventricular arc, or instead can be fixed to the actuator mechanism and receive direct traction from the actuator mechanism during the actuated state.

In one embodiment of the present invention, the ventricular-inflow ring is cyclically induced to generally flatten in the direction perpendicular to the separation of the leaflets of the atrioventricular valve, thereby facilitating closure of the atrioventricular valve during ventricular emptying, while allowing full expansion of the atrioventricular valve during ventricular filling.

In another embodiment, the septal splint is cyclically induced to narrow in the anterior-posterior direction and to bulge toward the lower pressure ventricle of the heart by a cyclical narrowing of the ventricular arc of the external framework element.

In another embodiment, the septal splint is cyclically twisted on its basal-to-apical axis by torsion of the limbs of the ventricular arc of the external framework element, thereby augmenting volume reduction in one or both ventricles.

The present invention, together with other and further objectives thereof, is set forth in greater detail in the following description, taken in conjunction with the accompanying drawings.

### BRIEF DESCRIPTION OF THE DRAWINGS

FIG. 1 is a frontal anterior perspective view of an exemplary natural heart with an external framework element around it in accordance with one aspect of the present invention;

FIG. 2 is a perspective view of a framework for interfacing with a natural heart in accordance with one aspect of the present invention;

FIG. 2A is another perspective view of a suitable framework;

FIG. 3 is a partial cross-sectional view of a natural heart with a septal splint made in accordance with the present invention placed within a natural heart;

FIG. 4 is a perspective view of the external framework element of the framework showing the basal arc flattening;

FIG. 5 is a perspective view of a heart with the left atrium removed just above the left ventricular inflow valve during ventricular filling;

FIG. 6 is a perspective view of a heart with the left atrium removed just above the left ventricular inflow valve during ventricular emptying;

FIG. 7 is a perspective view of the external framework element of the framework showing a change in angulation of the basal arc;

FIG. 7A is a side perspective view of the external framework element of the framework showing a change in angulation of the basal arc;

FIG. 8 is a posterolateral perspective view of the external framework element of the framework showing the anterior-posterior narrowing of the ventricular arc;

FIG. 9 is a cross-sectional view through the heart, the ventricular arc, and the septal splint at the level of the ventricles as they appear at the end of ventricular filling;

FIG. 10 is a cross-sectional view like in FIG. 9, through the heart, the ventricular arc, and the septal splint at the level of the ventricles as they appear during ejection;

FIG. 11 is a cross-sectional view through the heart, the ventricular arc, the septal splint, and the non-expansive sheath at the level of the ventricles as they appear at the end of ventricular filling;

FIG. 12 is a cross-sectional view through the heart, the ventricular arc, the septal splint, and the non-expansive sheath at the level of the ventricles as they appear during ventricular ejection;

FIG. 13 is a cross-sectional view through the heart, the ventricular arc, the septal splint, and the non-expansive sheath at the level of the ventricles as they appear during torsion of both limbs of the ventricular arc of the yoke;

FIG. 14 is a cross-sectional view through the heart, the ventricular arc, the septal splint, and the non-expansive sheath at the level of the ventricles showing the sheath margins of the non-expandable sheath attached to the actuator mechanism and during torsion of both limbs of the ventricular arc of the yoke;

FIG. 15 is a perspective view of the external framework element of the framework and septal splint as they appear at the end of ventricular filling; and

FIG. 16 is a perspective view of the external framework element of the framework and septal splint as they appear during torsion.

FIG. 17 is a cross-sectional view of a portion of the heart coupled with an embodiment of the invention during diastole;

FIG. 18 is a cross-sectional view similar to FIG. 17, but during systole.

FIG. 19 is a sectional view of one embodiment of a yoke in accordance with the invention.

FIG. 20 is a perspective view of a yoke in accordance with one embodiment of the invention.

FIG. 21 is a sectional view of another embodiment of a yoke in accordance with one embodiment of the invention.

FIG. 22 is a sectional view of another embodiment of a yoke in accordance with one embodiment of the invention.

### DETAILED DESCRIPTION OF THE INVENTION

The present invention utilizes framework elements of a heart framework, as disclosed in U.S. Pat. No. 5,957,977, in conjunction with other components, for actuating the heart in various manners to achieve the desired shaping and movement of the heart to assist its pumping function. Accordingly, an overview of a natural heart and the cardiac framework are provided herein for understanding the overall invention.

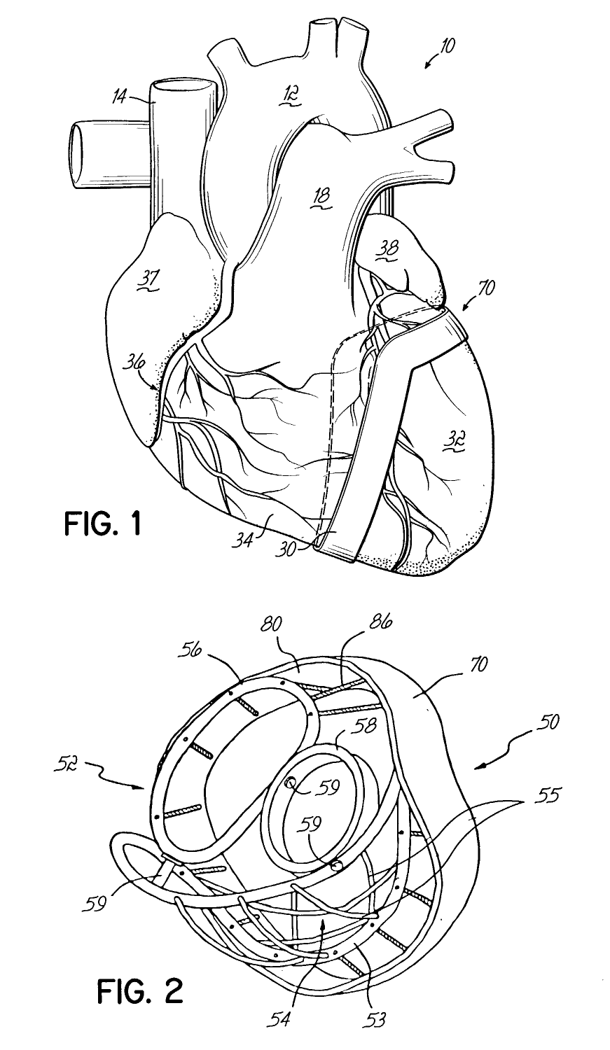

Referring now to the figures in detail wherein like numerals indicate the same elements throughout the views, a natural heart 10, generally indicated in FIG. 1, has a lower portion comprising two chambers, namely a left ventricle 32 and a right ventricle 34 which function primarily to supply the main force that propels blood through the circulatory system. The heart 10 also includes an upper portion having two chambers, a left atrium 38 and a right atrium 37 which primarily serve as an entryway to the ventricles and the system moving blood into the ventricles. The interventricular wall of cardiac tissue separating the left 32 and right 34 ventricles, respectively, is defined by an interventricular groove 30 on the exterior wall of the heart 10. The atrioventricular wall of the cardiac tissue separating the lower ventricular region from the upper atrial region is defined by the atrioventricular groove 36 on the exterior wall of the natural heart 10. Generally, the ventricles are in fluid communication with the atria through the atrioventricular valves. More specifically, the left ventricle is in fluid communication with the left atrium through the mitral valve while the right ventricle is in fluid communication with the right atrium through the tricuspid valve. Generally, the ventricles are in fluid communication with the circulatory system (i.e., the pulmonary and peripheral circulatory system) through semi-lunar valves. More specifically, the left ventricle is in fluid communication with the aorta of the peripheral circulatory system through the aortic valve while the right ventricle is in fluid communication with the pulmonary artery of the pulmonary circulatory system through the pulmonic valve.

The heart basically acts like a pump. The left and right ventricles are separate, but share a common wall, or septum. The left ventricle has thicker walls and pumps blood into the systemic circulation of the body. The pumping action of the left ventricle is more forceful than that of the right ventricle, and the associated pressure achieved within the left ventricle is also greater than in the right ventricle. The right ventricle pumps blood into the pulmonary circulation, including the lungs. During operation, the left ventricle fills with blood in the portion of the cardiac cycle referred to as diastole. The left ventricle then ejects any blood in the part of the cardiac cycle referred to as systole. The volume of the left ventricle is largest during diastole, and smallest during systole. The heart chambers, particularly the ventricles, change in volume during pumping. It is this feature to which the present invention is directed.

In accordance with one aspect, the invention utilizes a set of interconnected and actuatable intracardiac and extracardiac components, similar in shape, size, and interconnections to the combination of passive components collectively referred to as the “stint” and the “yoke” in U.S. Pat. No. 5,957,977. These components are the internal and external framework elements of the heart framework. In accordance with another aspect of the invention, one or more of the framework components are actuated, driven, or manipulated by an actuation system including an actuator mechanism that is coupled to the framework. In one embodiment, the internal framework elements include ring elements such as a first, or ventricular-inflow, ring and a second, or ventricular-outflow, ring, both of which are fixed to the fibrous annulus of their corresponding heart valves, namely the mitral and the aortic valves, respectively, in one preferred embodiment. The internal framework elements also include a septal splint, which is a net-like structure supporting the lower-pressure side of the interventricular septum, most likely the right ventricular side of the interventricular septum.

The external framework elements include a yoke which has at least one basal arc and one ventricular arc as further discussed below. These arcs can be separate connected parts or a single unitary part. In accordance with aspects of the invention, some or all of these components, and the connections between them, are specifically intended to be flexible, in that they are bent, twisted or otherwise acted upon by means of motion induced by an associated actuator mechanism of a actuator mechanism. That is, the framework elements are elastically deformed. “Flexibility” of these components and of their interconnections, as used herein, is intended to cover scenarios of desired elastic flexural rigidity or stiffness ranging from total flaccidity to near-complete rigidity. “Torsionability” of these components and their interconnections, as used herein, is intended to cover scenarios of desired torsional rigidity or stiffness ranging from total flaccidity to near-complete rigidity.

The invention operates by elastically deforming, bending and/or twisting of the individual framework components in response to movements of the external framework elements by an actuator mechanism. The deformed framework elements, in turn, deform portions of the heart. For example, when an actuator mechanism or device operates on the framework elements of the invention, the desired actuation and shaping of the heart and heart components occurs. Examples of this heart activation, together with potential reasons for doing so, are discussed below and illustrated in the accompanying Figures. Such examples are not meant to be limiting. Illustrations primarily show components directly affixed to the left ventricle, although they are equally applicable to components affixed to the right ventricle.

As noted, the present invention utilizes bending, flexing, reshaping, and general deforming of flexible framework components of the heart similar to those framework elements disclosed in U.S. Pat. No. 5,957,977. Greater detail regarding such a framework and its implantation within the human heart are set forth in that patent. However, the brief description of the various components of the framework is helpful in understanding aspects of the invention, and is therefore set forth herein.

A suitable framework is illustrated in FIG. 2 by reference numeral 50, which includes an internal framework element or elements 52 acting as an internal stint and an external framework element or elements 70 acting as a yoke 70 fixed to the internal framework element 52 by transmural cords 86 which extend through walls of the heart. A portion of the internal framework element 52 (i.e., a right ventricular splint 54) is sized and configured for placement within the interior volume of the natural heart 10, with element 53 generally alongside the right side of the interventricular septum. One embodiment of the internal framework element 52 utilizes a splint 54 which includes a generally triangular-shaped frame 53 that can be assembled from a plurality of interlocking struts, or are made of a single piece, part or all of which is flexible. Cords or strands extend across the frame 53 to form splint 54. Alternatively, the splint 54 might be formed using cords/strands without a frame 53. For example, FIG. 2A illustrates a framework 50a with rings 56, 58, a yoke 70, and cords 57, which form a splint 54a without a frame 53. The cords 57 are coupled directly to yoke 70.

The internal framework elements 52, also include two separate ring structures for positioning proximate the valve annuli of the left side of the heart. A first ring 56 is sized and configured for placement adjacent the atrioventricular valve annulus. For example, on the left side of the heart it would be placed suprajacent the mitral valve annulus in the left atrium. A second ring 58 is sized and configured for placement adjacent the semilunar valve annuli, for example, subjacent the aortic valve annuli in the left ventricle 32 on the left side of the heart. The first and second rings 56 and 58 and the septal splint 54 are attached at least to each other using connectors 59, (e.g. pins or other flexible or rigid connectors) to assist in maintaining their relative positions so that the first and second rings, 56 and 58 respectively, and the septal splint 54, are supported while the natural heart is being actuated in accordance with the invention.

As illustrated in FIG. 2, the framework 50 includes external yoke 70 for placement around a portion of the exterior surface or epicardium of a natural heart 10. The yoke 70 is generally stirrup-shaped and, in one regard, restricts free motion of the natural heart 10 when the framework is not actuated for actuation of the heart. The external framework element, or yoke 70, also may act as an anchor for an actuator mechanism of other heart wall actuation systems as set forth in U.S. patent application Ser. No. 09/850,554. Preferably, the yoke 70 is between about 1 and 2 cm wide, and is sized and configured for placement adjacent at least a portion of the atrioventricular groove 36, and simultaneously adjacent at least a portion of the anterior and posterior portions of the interventricular groove 30, and most preferably, adjacent at least a substantial portion of the anterior and posterior portion of the interventricular groove 30 as illustrated in FIG. 1.

General alignment of the external yoke 70 with the interior framework elements is maintained by at least one transmural cord 86, and preferably, a plurality of cords 86 that penetrate the walls of the natural heart 10 and connect to the internal framework element 52 and one or more of the rings 56, 58. In the embodiment of the splint 54 which does not use a frame, the cords 86 would couple the yoke directly to the strands of the splint, as illustrated in FIG. 2A.

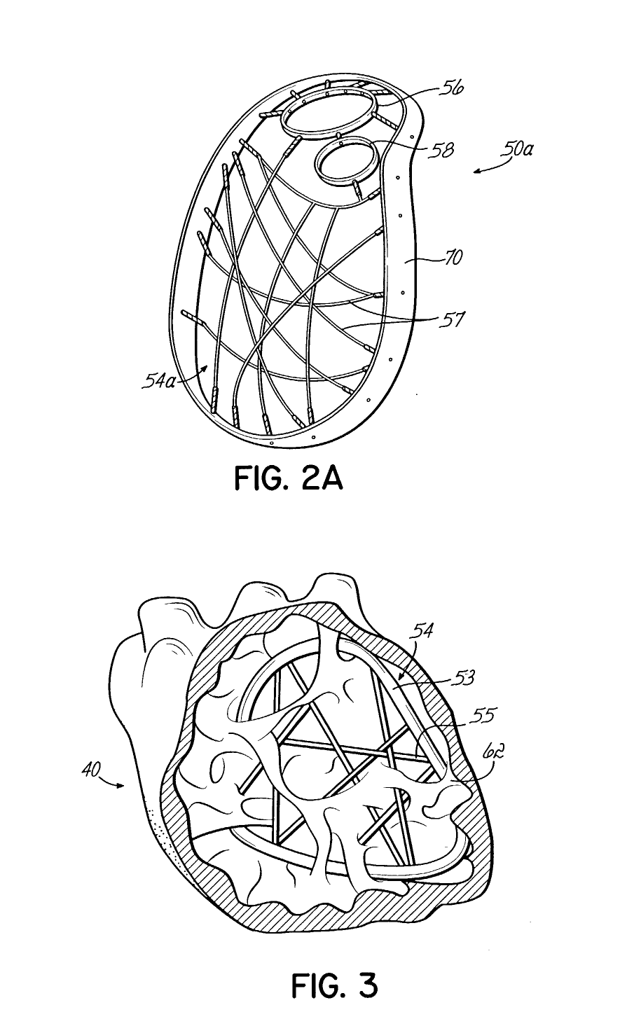

FIG. 3 further illustrates a septal splint 54 which includes one or more strands of sutures 55 affixed to the frame 53 through loops positioned on the frame 53, preferably the loops are affixed to the inner portion of frame 53, and more preferably at about 1.5 cm intervals. The splint 54 can take the form of a netlike configuration, or a snowshoe-like shaped configuration to brace or stabilize one side of the septum of the heart, without distortion of the chordae structures of the heart.

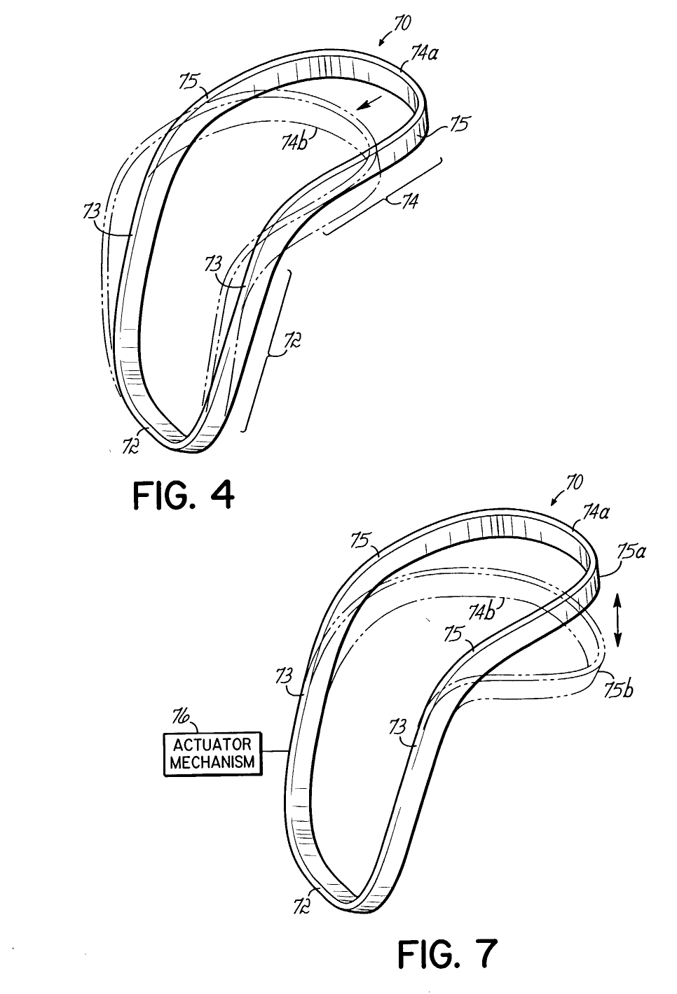

As noted above, some or all of the framework components or elements of an embodiment of the invention are configured for being deformed, such as by bending or twisting. For example, the framework elements might be elastic and may be deformed. Alternatively, the framework elements might be configured to have movable sections, such as hinged or sliding sections which move so the framework element may be deformed. In accordance with one aspect of the present invention, individual elements of the framework, and the connections between them, are deformed in response to the movements of an external element of the frame, such as the yoke 70. In accordance with one embodiment of the invention, and referring to FIG. 4, the yoke 70 includes multiple arc portions which are coupled together. Specifically, yoke 70 includes a basal arc or arc portion 74 and a ventricular arc or arc portion 72. As noted above, yoke 70 might be formed as a unitary structure. Alternatively, the basal arc 74 and ventricular arc 72 are formed separately and coupled together for use in the invention. For example, the arcs may be hingedly or slidably coupled together.

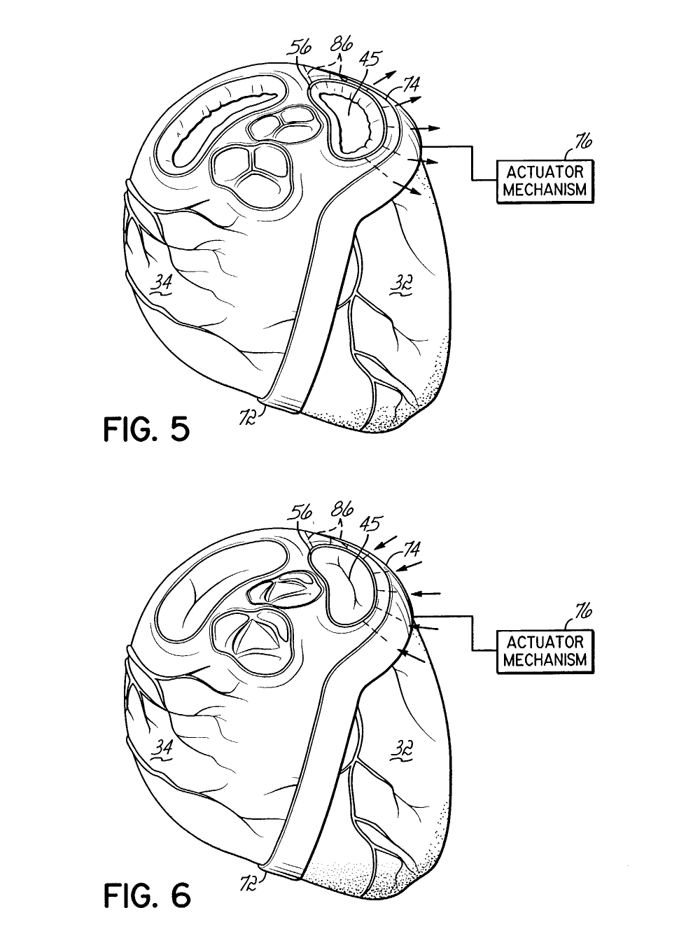

In one embodiment of the invention, the ventricular-inflow ring, such as ring 56 is cyclically induced to be deformed and flexed, such as to generally flatten, during the ejection phase of the pumping heart. The basal arc 74 flattens in a direction perpendicular to the separation of major leaflets or cusps of the valve encircled by the ring to thereby flatten the ring and affect the valve. In one embodiment of the invention, this flattening is accomplished by flattening of the basal arc of the yoke utilizing an attached actuator mechanism as illustrated in FIGS. 5 and 6.

As illustrated in FIG. 4, when the external framework element or yoke 70 is made of a pliable, flexible material, the basal arc 74 is flattened by the action of a suitable actuator mechanism acting on the external element as illustrated by various motion arrows in FIGS. 4, 5, and 6. Because of local differences in the balance between yoke flexibility and actuator-induced forces, the ventricular arc 72 of the yoke 70 may remain generally unaffected while the basal arc is moved from a position at 74a to a position at 74b (see FIG. 4).

FIGS. 5 and 6 sequentially depict this basal arc flattening and its subsequent effect on the ring 56 and the mitral valve leading to the left ventricle. While embodiments herein are shown as acting on the left ventricle and associated valves, the invention can be applied to the right side of the heart as well. Specifically, the invention might be applied to a tricuspid (right ventricular inflow) ring and valve as well. In FIGS. 5 and 6, the heart is shown with the left atrium removed just above the left ventricular inflow valve. FIG. 5 depicts the activity in accordance with an embodiment of the invention during ventricular filling, and FIG. 6 depicts such activity during ventricular emptying. This basal arc 74b flattening or flexing depicted in FIG. 6 in turn causes the ventricular-inflow ring 56 to be flattened or flexed in the direction perpendicular to the separation of the leaflets of the left atrioventricular, or mitral, valve 45. This facilitates closure of the valve during ventricular ejection or emptying by bringing together (apposition) of the major leaflets of the valve 45, while allowing full expansion of the base of the ventricle 32 during ventricular filling, as seen in FIG. 5. This action is cyclically induced by the actuator mechanism 76 of the actuation system. As noted, although a mitral ring and valve are illustrated, this action may be applied to a tricuspid, or right ventricular ring and valve as well.

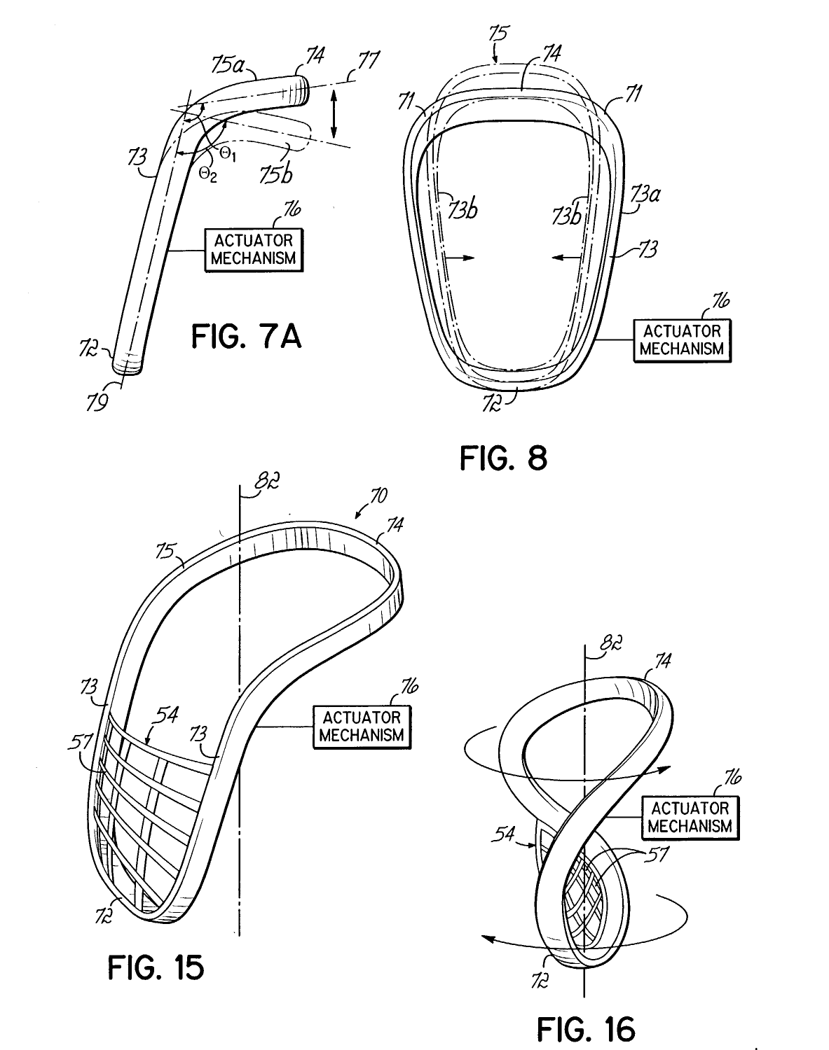

In another embodiment of the invention, the angulations between the rings 56, 58 and the septal splint 54 of the internal framework elements are cyclically altered by changes or variations in angulation between the basal and ventricular arcs 74, 72 of the external framework element or yoke 70. This change in angulation is induced by an attached actuator mechanism 76. FIG. 7 shows a perspective view and FIG. 7A shows a side view of the external framework element deformation needed for the above example. The plane 77 defined generally by the basal arc 74 is altered to change angulation with respect to the plane 79 defined by the ventricular arc 72. Referring to FIG. 7A, the angle θ₂ after such change in angulation, is less than θ₁ before the angulation. During angulation, the limbs or legs 75 of the basal arc 74 move between positions 75a and 75b, as shown, while the limbs or legs 73 of the ventricular arc 72 are maintained in a generally similar position whether relaxed or actuated.

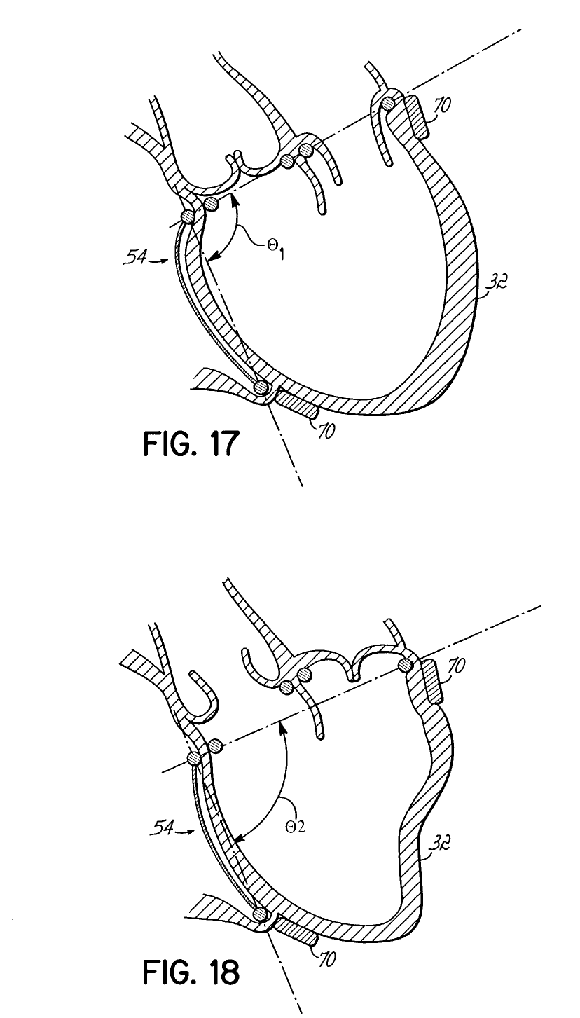

As illustrated in FIGS. 17 and 18, cross-sectional views of portions of the heart are shown in the long axis of the heart. FIG. 17 illustrates a portion of the heart (particularly, the left ventricle) during filling (diastole) showing a position of the framework elements in the relative position.

FIG. 18 illustrates a cross-sectional view of the heart similar to FIG. 17 during ejection or emptying (systole) and showing a position of the framework elements which might be imposed in accordance with an embodiment of the invention wherein the angulation between the two arcs of the yoke is modified as discussed above with respect to FIGS. 7 and 7A.

As shown in FIG. 8, the yoke may be flexibly deformed such that the various arcs are narrowed to narrow the septal splint in the anterior-posterior direction. This narrowing may be induced by an attached actuator mechanism 76, which varies the distance between the respective limbs 73 and 75 of the ventricular arc 72 and the basal arc 74. FIG. 8 shows the yoke flexed or narrowed from position 73a to position 73b. This narrowing will allow the septal splint 54 to deflect or bulge toward the lower pressure side (i.e. generally the right ventricle side) during emptying of the heart, thus transferring some of the volume reduction from the high pressure ventricle to the low pressure ventricle. This transfer of volume reduction will reduce, or possibly eliminate, the need for a separate means of low-pressure ventricle volume alteration, even when the natural function of that ventricle is sufficiently depressed as to require mechanical assistance.

The volume reduction action may be facilitated by the presence of a flexible, non-expandable external sheath 80 applied to the surface of the low-pressure ventricle. The sheath 80 may be utilized and implemented by induced torsion or movement of the limbs 73 of the ventricular arc 72, by direct traction of the margins of the sheath 80, or both. An example of such a sheath is illustrated in FIGS. 11-14.

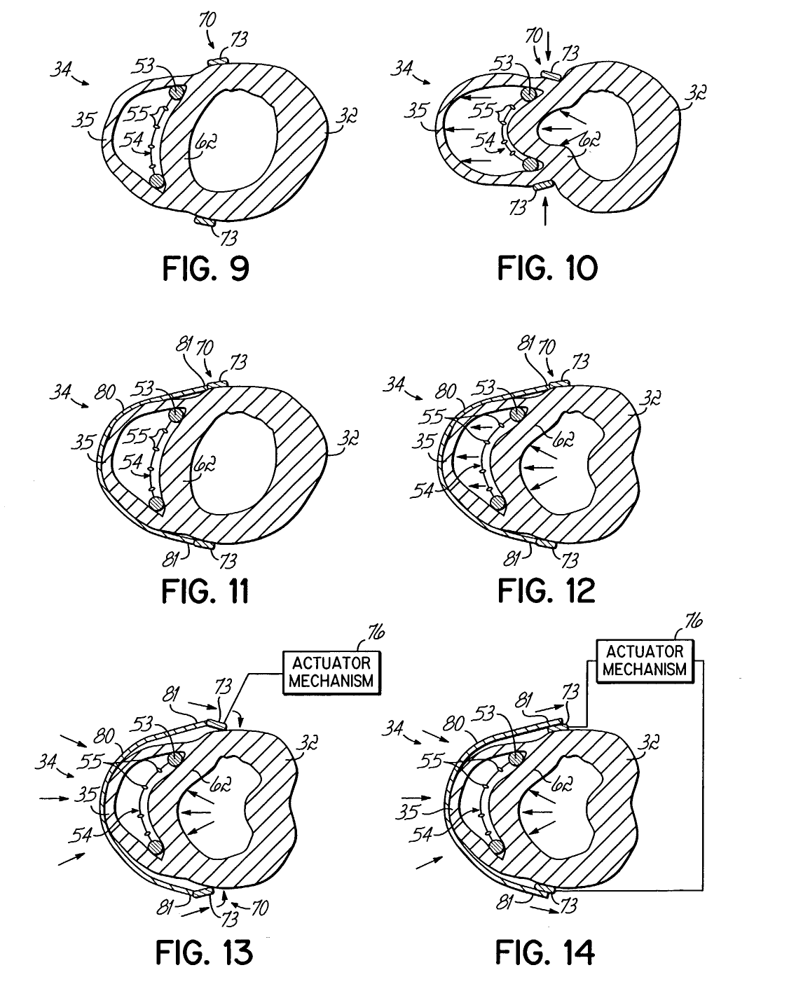

FIGS. 9 through 14 are cross-sectional views through a heart at the ventricular level illustrating the actuation of various embodiments of the invention, utilizing narrowing of the ventricular arc, using a non-expandable sheath 80, and a combination of both components. FIG. 9 depicts the ventricles, the limbs 73of the ventricular arc 72, and the septal splint 54 as they might appear at the end of ventricular filling (diastole). FIG. 10 illustrates the same components and heart chambers as they might appear at the end of ventricular ejection or emptying (systole) when utilizing narrowing of the ventricular arc as noted above with respect to FIG. 8. The free wall 35 of the low-pressure ventricle, (here, the right ventricle 34) may bulge outwardly, reducing the net volume reduction in that ventricle. This bulging has two potential causes. First, there is reduced separation between the anterior and posterior margins of the free wall due to narrowing of the ventricular arc from movement of the limbs 73 of the ventricular arc 72. Secondly, there is stretching of the free wall 35 of the ventricle 34 due to increased chamber pressure in the right ventricle.

To address the distention or bulging of the wall 35 of the right ventricle 34, one embodiment of the present invention utilizes a sheath 80 which is positioned around the ventricle wall. In one embodiment, the sheath is generally non-expandable. FIG. 11 illustrates a cross-sectional view similar to that of FIG. 9, except that a generally passive, generally non-expandable sheath 80 is positioned around the outside of the wall 35 of the low-pressure ventricle 34 (i.e. generally, but not necessarily, the right ventricle as illustrated herein). The margins 81 of the sheath 80 are fixed to the limbs 73 of the ventricular arc 72 of the yoke 70, and may possibly also be fixed to the arc 74 of yoke 70.

FIG. 12 is a cross-sectional view similar to FIG. 11 during systole, again showing bulging of the interventricular septum 62 towards the low-pressure ventricle 34. However, the stretching of the free wall 35 of ventricle 34, due to increased chamber pressure, is not as prominent as shown in FIG. 10 or may even be generally non-existent, since the wall is prevented from stretching significantly by the generally non-expandable sheath 80. This lessens the loss of net volume reduction which would be associated with the expanded or stretched wall 35.

FIG. 13 illustrates a sheath 80 similar to that of FIGS. 11 and 12, but which is actually drawn in or tightened during systole by torsion forces produced by an actuator mechanism coupled to the yoke 70. The torsion of the two limbs 73 and the resultant narrowing of the ventricular arc 72 of the yoke 70 further reduces the volume of the low-pressure ventricle 34. This reduction in volume has a synergistic effect with the bulging of the ventricular septum 62. That is, the sheath margins are fixed to the limbs of the ventricular arc and the sheath is tensed or drawn when the limbs of the arc are manipulated, such as by twisting and/or rotation.

FIG. 14 illustrates a sheath 80 in which the margins 81 of the sheath 80 are directly attached to a suitable actuator mechanism 76. The actuator mechanism as shown in FIG. 14 is operable to be movable between an actuated state and a relaxed state to draw the sheath margins in the direction of the reference arrows shown around sheath 80. That is, attaching the sheath margins 81 to the actuator mechanism 76 will apply traction to the sheath to pull the sheath 80 into the direction opposite to the expanding wall 35 of the low-pressure ventricle 34. This action will serve to further augment the ability of the non-expandable sheath 80 to prevent stretching of the free wall 35 of the low-pressure ventricle 34, thereby further lessening the loss of net volume reduction in that ventricle associated with the stretched wall 35.

FIGS. 15 and 16 illustrate an alternative embodiment of the invention. In such an embodiment, torsional forces are introduced on a heart chamber, such as a left ventricle, by twisting the yoke 70 along an axis, indicated by 82 in FIGS. 15 and 16. More specifically, FIG. 15 illustrates a yoke 70 with the and suture strands or cords 57 of a septal splint 54 in the non-actuated or relaxed position. When the actuator mechanism actuates the ventricular arc 72 of the yoke 70, the arc and the septal splint 54 are twisted around the axis 82 which might be considered a basal-to-apical axis. In accordance with the embodiments of FIGS. 15 and 16, the torsional twisting of yoke 70 and splint 54 may be utilized to augment, or possibly replace, the volume reduction operations which are associated with indentation of the lateral wall of the ventricular chamber or other heart chamber. The torsional twisting of the yoke 74 and splint 54 provides a “wringing” action on the ventricle, such as a left ventricle. FIG. 16 illustrates the twisting action of an actuated yoke in accordance with the embodiment of the invention.

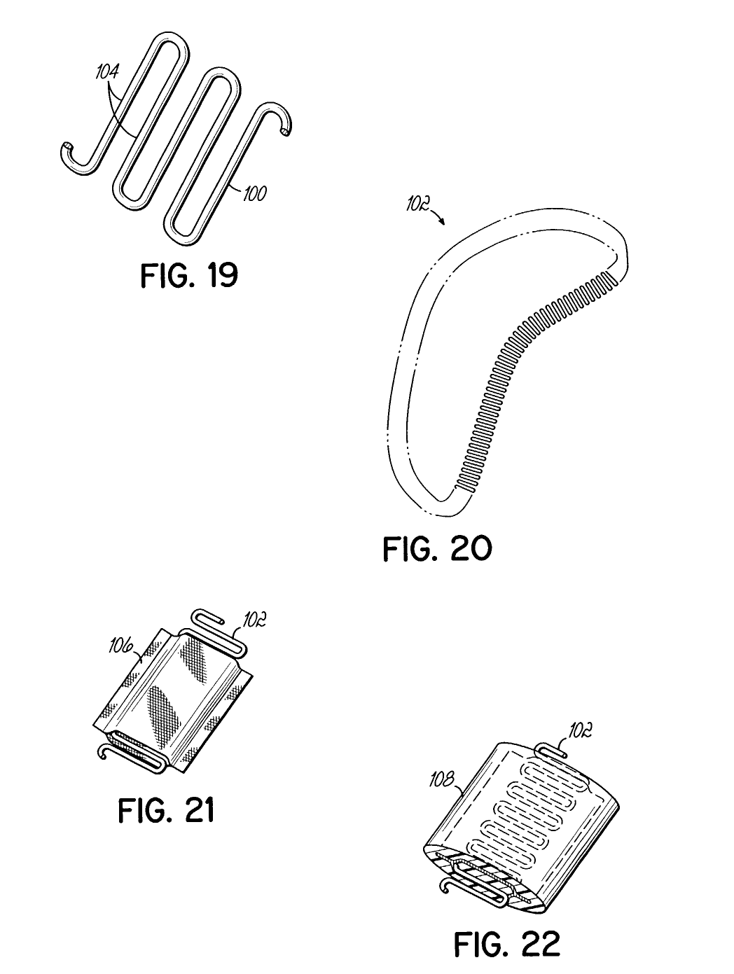

FIG. 19 illustrates the sectional view of one possible embodiment of a yoke structure in accordance with the principles of the present invention. Specifically, to construct a yoke that is both flexible and torsionable, a wire 100 is formed in a “zig-zag” pattern to form a flat spring as illustrated in FIG. 20. The yoke 102 is formed with a suitable material, such as stainless steel, CP titanium, or a shape-memory material wherein individual sections 104 of the wire define the width of the yoke 102.

The yoke 102 might utilize the flat spring structure alone or the spring structure may be utilized in combination with a jacket as illustrated in FIG. 21. Specifically, a jacket 106 is formed of a biocompatible fabric such as polyester or another suitable fabric. The jacket 106 may be knitted, woven or otherwise formed to surround the flat spring yoke 102.

In still another alternative embodiment as illustrated in FIG. 22, a material may be molded around the flat spring yoke 102. For example, a molded jacket or sheath 108 might be formed around yoke 102. The jacket could be made of a soft elastomeric material, such as silicone rubber or polyurethane. Such an elastomeric body or jacket 108 would present a soft, smooth external surface and contour to any contacting tissue. While the bulk of the load of any flexing or twisting of yoke 102 would be borne by the flat spring structure, the jackets or other coverings 106, 108 would provide a material of intermediate modulus between the hard material of the yoke 102 and the soft material of the jackets 106, 108. This would lessen the likelihood of either tearing of the jackets or of delamination between the yoke 102 and the jackets 106, 108.

As discussed above, one or more actuator mechanisms or curvatures/shape limiting elements may need to be attached at various points to the yoke 102. This might be accomplished either by leaving intervals in which the zig-zag wire spring is exposed (e.g., if clad in a jacket) to allow bolting, clamping, welding, cementing or other fixation, or by interposing segments in the yoke that are specifically designed for such attachment (e.g., short metallic plates which have ends attached to yoke segments).

The advantage of the present invention is that greater control of deformation patterns can be induced in the ventricles by variation in degrees of elastic flexural rigidity of components. Such control enables simultaneous optimization of strain and strain rates in all regions of the heart tissue. It also optimizes the alleviation of stress and strain induced by the actuator mechanism in the intracardiac and extracardiac components, as well as optimizing flow patterns induced in the ventricle during filling and ejection. It also optimizes ventricular ejection volume, which can be life-saving to an individual with a failing heart.

While the present invention has been illustrated by the description of the embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of applicant's general inventive concept.

What is claimed is:

1. An actuation system for assisting the operation of a natural heart and comprising:
a framework element configured to be coupled with a portion of the heart;
the framework element being configured for being deformed;
an actuator mechanism coupled to the framework element and operable for deforming the framework element for varying the shape of the heart.

2. The actuation system of claim 1 further comprising:
an internal framework element configured to be positioned within the interior volume of the heart and to be coupled to at least a portion of the internal tissue of the heart; and
an external framework element configured to be positioned proximate an exterior surface of the heart, the internal and external framework elements being coupled together,
at least one of the elements of the framework being configured for being deformed;
the actuator mechanism coupled to the at least one of the framework elements, and operable for deforming at least one framework element for varying the shape of the heart.

3. The actuation system of claim 1, wherein the actuator mechanism is selectively movable between an actuated state and a relaxed state and operable, when actuated, for deforming the framework element.

4. The actuation system of claim 2 further comprising at least one cord component configured for extending through the tissue of the heart and for coupling together the external and internal framework elements.

5. The actuation system of claim 2, wherein both the internal and external framework elements are flexible.

6. The actuation system of claim 1 including multiple framework elements.

7. The actuation system of claim 2 including multiple internal framework elements comprising:
a first ring configured for placement adjacent one of the annuli of the heart; and
a second ring configured for placement adjacent another of the annuli of the heart.

8. The actuation system of claim 2, the internal framework element including a ring configured for placement adjacent one of the annuli of the heart.

9. The actuation system of claim 2, the internal framework element including a septal splint configured for coupling to a portion of a septal wall between chambers of the heart.

10. The actuation system of claim 6, wherein the multiple framework elements further comprise a septal splint configured for coupling to a portion of a septal wall between chambers of the heart.

11. The actuation system of claim 7, wherein the multiple internal framework elements further comprise at least one connector joining the first ring and the second ring.

12. The actuation system of claim 1, wherein the framework element comprises a yoke having a basal arc and a ventricular arc configured to extend at an angle to the basal arc.

13. The actuation system of claim 12 wherein the basal arc of the yoke is flexible for being flattened by the actuator mechanism.

14. The actuation system of claim 13 including multiple framework elements comprising a flexible ring configured for placement adjacent one of the annuli of the heart, the ring coupled to the yoke and operable for being flexed when the basal arc is flattened.

15. The actuation system of claim 12 wherein the yoke is flexible for flexing generally between the basal arc and the ventricular arc, the actuator mechanism being operable for varying the angle between the basal arc and the ventricular arc.

16. The actuation system of claim 12 wherein the yoke includes opposing limbs, the yoke being flexible for varying the distance between the limbs and the actuator mechanism operable for flexing the and varying the limb distance.

17. The actuation system of claim 16 including multiple framework elements comprising a flexible septal splint configured for coupling to a portion of a septal wall between chambers of the heart, the septal splint spanning between the limbs of the ventricular arc and flexing when the yoke is flexed.

18. The actuation system of claim 12, further comprising a sheath configured for placement adjacent a wall of a ventricle of the heart.

19. The actuation system of claim 12 wherein the sheath is generally non-expandable.

20. The actuation system of claim 18, wherein the sheath margins are fixed to the limbs of the ventricular arc, the sheath being operable for flexing when the ventricular arc is flexed.

21. The actuation system of claim 18, wherein the sheath margins are fixed to the limbs of the ventricular arc, the sheath operable for being tensed when limbs of the arc are one of twisted and rotated on their long axes.

22. The actuation system of claim 18, wherein the sheath margins are fixed to the actuator mechanism, the actuator mechanism being selectively movable between an actuated state and a relaxed state and operable for applying direct traction to the sheath margins during the actuated state.

23. The actuation system of claim 12 wherein the ventricular arc includes opposing limbs, the ventricular arc being flexible for being twisted on a vertical axis, and the actuator mechanism operable for flexing the ventricular arc and twisting the limbs around the axis.

24. The actuation system of claim 23 including multiple framework elements comprising a flexible septal splint configured for coupling to a portion of a septal wall between chambers of the heart, the septal splint spanning between the limbs of the ventricular arc and flexing when the ventricular arc is flexed.

25. The actuation system of claim 24, further comprising a sheath configured for placement adjacent a wall of a ventricle of the heart.

26. The actuation system of claim 25, wherein the sheath margins are fixed to the limbs of the ventricular arc, the sheath being operable for flexing when the ventricular arc is flexed.

27. The actuation system of claim 25, wherein the sheath margins are fixed to the actuator mechanism, the actuator mechanism being selectively movable between an actuated state and a relaxed state and operable for applying direct traction to the sheath margins during the actuated state.

28. The actuation system of claim 12 wherein the yoke comprises a flat spring structure.

29. The actuation system of claim 28 further comprising a jacket positioned on the yoke.

30. The actuation system of claim 29 wherein the jacket is one of a fabric and a molded material.

31. A method of assisting the pumping function of the natural heart comprising:
interfacing the natural heart with an actuation system, the actuation system comprising an actuator mechanism and a framework element for coupling with a portion of the heart, the framework element being configured for being deformed;
coupling the actuator mechanism to the framework element; and
deforming the framework element by moving the actuator mechanism for varying the shape of the heart.

32. The method of claim 31, wherein the actuator mechanism is selectively movable between an actuated state and a relaxed state and operable, when actuated, for deforming the framework element.

33. The method of claim 31 further comprising:
coupling an internal framework element to a portion of the internal tissue of the heart; and
coupling an external framework element to portion of the external tissue of the heart; and
deforming at least one of the framework elements with the actuator mechanism.

34. The method of claim 33, wherein both the internal and external framework elements are flexible.

35. The method of claim 31, wherein the framework element comprises a yoke having a basal arc and a ventricular arc configured to extend at an angle to the basal arc.

36. The method of claim 31, wherein the basal arc is flexible for being flattened by the actuator mechanism.

37. The method of claim 36 including multiple framework elements comprising a flexible ring configured for placement adjacent one of the annuli of the heart, the ring coupled to the yoke and operable for being flexed when the basal arc is flattened.

38. The method of claim 35, wherein the yoke is flexible for flexing generally between the basal arc and the ventricular arc, the actuator mechanism being operable for varying the angle between the basal arc and the ventricular arc.

39. The method of claim 35 wherein the yoke includes opposing limbs, the yoke being flexible for varying the distance between the limbs and the actuator mechanism operable for flexing the yoke and varying the limb distance.

40. The method of claim 39 including multiple framework elements comprising a flexible septal splint configured for coupling to a portion of a septal wall between chambers of the heart, the septal splint spanning between the limbs of the yoke and flexing when the ventricular arc is flexed.

41. The method of claim 31, the actuation system further comprising a sheath configured for placement adjacent a wall of a ventricle of the heart.

42. The method of claim 41 wherein the sheath is generally non-expandable.

43. The method of claim 41, wherein the sheath margins are fixed to the limbs of the ventricular arc, the sheath being operable for flexing when the ventricular arc is flexed.

44. The method of claim 41, wherein the sheath margins are fixed to the limbs of the ventricular arc, the sheath being operable for being tensed when limbs of the arc are one of twisted and rotated on their long axes.

45. The method of claim 41, wherein the sheath margins are fixed to the actuator mechanism, the actuator mechanism being selectively movable between an actuated state and a relaxed state and operable for applying direct traction to the sheath margins during the actuated state.

46. The method of claim 35 wherein the ventricular arc includes opposing limbs, the ventricular arc being flexible for being twisted on a vertical axis, and the actuator mechanism operable for flexing the ventricular arc and twisting the limbs around the axis.

47. The method of claim 46 including multiple framework elements comprising a flexible septal splint configured for coupling to a portion of a septal wall between chambers of the heart, the septal splint spanning between the limbs of the ventricular arc and flexing when the ventricular arc is flexed.

48. The method of claim 47, further comprising a sheath configured for placement adjacent a wall of a ventricle of the heart.

49. The method of claim 48, wherein the sheath margins are fixed to the limbs of the ventricular arc, the sheath being operable for flexing when the ventricular arc is flexed.

50. The method of claim 48, wherein the sheath margins are fixed to the actuator mechanism, the actuator mechanism being selectively movable between an actuated state and a relaxed state and operable for applying direct traction to the sheath margins during the actuated state.
